(19) United States
(12) Patent Application Publication
(10) Pub. No.: US 2026/0265664 A1
(43) Pub. Date: Sep. 10, 2026

(54) FABRICATION METHODS FOR DETERMINISTIC MECHANOPORATION GENE DELIVERY DEVICES AND SYSTEMS

(71) Applicant: Basilard BioTech, Inc., Temecula, CA (US)

(72) Inventors: James ONUFFER, Temecula, CA (US); Faysal AHMED, Temecula, CA (US); Dennis YOST, Temecula, CA (US); Brynley LEE, Temecula, CA (US)

(21) Appl. No.: 19/350,437

(22) Filed: Oct. 6, 2025

Related U.S. Application Data
(63) Continuation of application No. PCT/US2024/023130, filed on Apr. 4, 2024.
(60) Provisional application No. 63/494,964, filed on Apr. 7, 2023.

(51) Int. Cl. C12M 1/42; B01L 3/00; C12M 1/32

(52) U.S. Cl. CPC C12M 35/04; B01L 3/5085; C12M 23/12; B01L 2200/0647; B01L 2200/12; B01L 2300/0829; B01L 2300/12

(57) ABSTRACT

Described are methods for the fabrication of devices for delivery of a payload to a cell, including gene transfection of the cell. Such fabrication methods involve selective material deposition for control of feature properties of the device. Such device features include projections (e.g., needles) and capture site (e.g., wells) perimeters.

### CROSS REFERENCE TO OTHER APPLICATION(S)

This application is a continuation of International Application No. PCT/US2024/023130, filed Apr. 4, 2024, which claims the benefit of U.S. Provisional Application No. 63/494,964, filed Apr. 7, 2023, all of which are incorporated herein in their entireties by reference.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BACKGROUND

Intracellular delivery of exogenous materials into large populations of suspension cells is required for a growing number of life science applications. Therefore, there is a need for devices, systems and methods allowing for high throughput and efficient intracellular delivery of exogenous materials while maintaining viability of the cells after delivery. Gene transfection is one such application.

### SUMMARY

In an aspect, provided is a method for fabricating a gene delivery device, the method comprising the operations of: etching a plurality of feature regions on a front side of a substrate, the substrate comprising a substrate material; depositing a feature material on the front side of the substrate; etching a portion of the deposited feature material on the front side of the substrate, thereby forming a front side surface (e.g., front side device surface); etching the front side of the substrate to form a plurality of wells; etching the front side of the substrate to form a plurality of ports; and etching a back side of the substrate to form at least one sub-array trench (e.g., back side aspiration port), and wherein the substrate material is different from the feature material. In some embodiments, the depositing comprises atomic layer deposition (ALD), sputtering, physical vapor deposition (PVD), chemical vapor deposition (CVD), plasma-enhanced chemical vapor deposition (PECVD), electron beam evaporation, or thermal evaporation. In some embodiments, the depositing comprises depositing parameters. In some embodiments, the etching comprises etching the front side of the substrate until the deposited feature material is only present within the plurality of feature regions. In some embodiments, the feature material is an electrically conductive material. In some embodiments, the feature material comprises silicon dioxide, aluminum dioxide, hafnium dioxide, titanium dioxide, chrome oxide, chromium oxide, zirconium oxide, zinc oxide, silicon nitride, tungsten, platinum, boron nitride, porous silicon or titanium nitride. In some embodiments, the etching comprises dry etching (e.g., plasma etching), chemical mechanical polishing (CMP), or wet etching. In some embodiments, the wet etching comprises wet etching parameters. In some embodiments, the wet etching parameters comprise an etchant, an etchant concentration, an etch time, an etch temperature or a combination thereof. In some embodiments, the etchant comprises sodium hydroxide or buffered oxide etch. In some embodiments, the etching comprises dry etching (e.g., plasma etch) parameters. In some embodiments, the dry etching parameters comprise a power, at least one gas, a flow rate of each gas of the at least one gas; an etch process pressure, an etch time, an etch temperature, or a combination thereof. In some embodiments, the etching the front side of the substrate to form the plurality of wells comprises selective and isotropic wet etching through openings of a well-mask. In some embodiments, the etching the front side of the substrate to form the plurality of ports comprises anisotropically etching the front side of the substrate. In some embodiments, the etching the back side of the substrate to form the at least one sub-array trench comprises anisotropically etching the back side of the substrate. In some embodiments, the anisotropically etching comprises dry etching through a port-mask. In some embodiments, the port-mask is formed by a process comprising photolithography. In some embodiments, the plurality of ports comprises a plurality of aspiration vias. In some embodiments, each feature region of the plurality of feature regions is configured to form a feature of a plurality of features of the device. In some embodiments, each feature of the plurality of features comprises a projection. In some embodiments, the method further comprises sharpening the projection. In some embodiments, the sharpening comprises argon sputter etching, ion milling or thermally oxidizing followed by buffered oxide etching. In some embodiments, forming the feature region comprises anisotropically etching the feature region with a dry etch process through a feature region-mask. In some embodiments, the feature region-mask is formed by photolithography. In some embodiments, the projection comprises a projection length. In some embodiments, each well of the plurality of wells comprises a well depth. In some embodiments, the projection length and well depth comprise a projection length to well depth ratio. In some embodiments, the projection comprises a tip. In some embodiments, the projection tip comprises a recess depth, the recess depth comprising the distance between the extreme end of the projection tip and the front side surface. In some embodiments, the projection comprises a base. In some embodiments, projection length and recess depth are determined by the amount of feature material deposited. In some embodiments, the projection length and recess depth are controlled by parameters of the depositing, the etching, the sharpening, or a combination thereof. In some embodiments, the feature comprises a well boundary perimeter (e.g., outline). In some embodiments, the device comprises a plurality of capture sites. In some embodiments, each of the capture sites of the plurality of capture sites comprises at least one of the plurality of aspiration vias. In some embodiments, the plurality of capture sites are arranged in at least one sub-array. In some embodiments, the least one sub-array is arranged in an array of sub-arrays. In some embodiments, each aspiration via of the plurality of aspiration vias comprises an aspiration via channel. In some embodiments, the substrate comprises a feature layer, an insulator layer, a handle layer, or a combination thereof. In some embodiments, the etching the back side of the substrate to form the least one sub-array trench comprises aligning the least one sub-array trench on the back side of the substrate to the least one sub-array on the front side of the substrate. In some embodiments, the etching the back side of the substrate to form the least one sub-array trench comprises etching through the handle layer and the insulator layer, thereby allowing the plurality of aspiration via channels to be connected with the least one sub-array trench. In some embodiments, the etching comprises plasma etching, wet etching or a combination thereof. In some embodiments, the method further comprises functionalizing the projection with a payload. In some embodiments, the payload comprises a biomolecule. In some embodiments, the substrate material comprises a semiconductor. In some embodiments, the substrate comprises silicon, silicon oxide, or a combination thereof. In some embodiments, the feature layer comprises silicon. In some embodiments, the insulator layer comprises silicon dioxide. In some embodiments, the handle layer comprises silicon. In some embodiments, the operations of: etching a plurality of feature regions on a front side of a substrate, the substrate comprising a substrate material; depositing a feature material on the front side of the substrate; etching a portion of the deposited feature material on the front side of the substrate, thereby forming a front side surface (e.g., front side device surface); etching the front side of the substrate to form a plurality of wells; etching the front side of the substrate to form a plurality of ports; and etching a back side of the substrate to form at least one sub-array trench (e.g., back side aspiration port), are performed in any order. In some embodiments, the operation of etching the back side of the substrate to form the sub-array trench, occurs at the beginning or at the end of the process flow prior to oxide removal. In some embodiments, the operation of etching the back side of the substrate to form the sub-array trench is performed prior to: etching a plurality of feature regions on a front side of a substrate, the substrate comprising a substrate material; depositing a feature material on the front side of the substrate; etching a portion of the deposited feature material on the front side of the substrate, thereby forming a front side surface (e.g., front side device surface); and etching the front side of the substrate to form a plurality of wells. In some embodiments, the operation of etching the back side of the substrate to form the sub-array trench is performed after: etching a plurality of feature regions on a front side of a substrate, the substrate comprising a substrate material; depositing a feature material on the front side of the substrate; etching a portion of the deposited feature material on the front side of the substrate, thereby forming a front side surface (e.g., front side device surface); and etching the front side of the substrate to form a plurality of wells. In some embodiments, the projection does not move relative to the substrate. In some embodiments, the projection does not move relative to the front side surface of the substrate.

In an aspect, provided is a method for fabricating a gene delivery device, the method comprising: etching a plurality of feature regions on a front side of a substrate, the substrate comprising a substrate material; depositing a feature material on the front side of the substrate; removing a portion of the deposited feature material on the front side of the substrate, thereby forming a front side surface (e.g., front side device surface); etching the front side of the substrate to form a plurality of wells; etching the front side of the substrate to form a plurality of ports; and etching a back side of the substrate to form at least one sub-array trench (e.g., back side aspiration port), and wherein the substrate material is different from the feature material. In some embodiments, the removing comprises lifting-off the portion of the deposited feature material on the front side of the substrate. In some embodiments, the lifting-off comprises dissolving a lift-off layer residing between the substrate and a mask defining the plurality of feature regions.

In an aspect, provided is a method for fabricating a gene delivery device, the method comprising: etching a plurality of projection regions on a front side of a substrate; depositing a projection material on the front side of the substrate; etching a layer comprising a portion of the deposited projection material on the front side of the substrate; etching the front side of the substrate to form a plurality of wells; etching the front side of the substrate to form a plurality of ports; sharpening a projection formed after the operation of etching the front side of the substrate to form a plurality of wells; and etching the back side of the substrate to form at least one sub-array trench, and wherein, the projection does not move relative to the substrate.

In an aspect, provided is a method for fabricating a gene delivery device, the method comprising: etching a plurality of wells on a front side of a substrate, the etching occurring within a top layer of the substrate, the top layer comprising an oxide material; etching a plurality of aspiration vias within each well of the plurality of wells on the front side of the substrate; and anisotropically etching a back side of the substrate to form a sub-array trench, and wherein the projection does not move relative to the substrate.

In an aspect, is a device comprising: a surface; and a capture site, the capture site comprising: a well having a well boundary shape at the surface and having a well depth, the well configured to support a cell; and a projection extending from the bottom of the well toward the surface of the substrate, and wherein the projection does not move relative to the substrate.

In an aspect, is a device comprising: a surface; and a capture site, the capture site comprising: a well having a well boundary shape at the surface and having a well depth, the well configured to support a cell; and a projection extending from the bottom of the well toward the surface of the substrate, the projection comprising a projection length, and wherein the substrate material is different than the feature material.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIGS. 1A-1F show various operational mechanisms for a gene delivery device in accordance with some embodiments.

FIG. 2 shows a gene dene delivery device in accordance with some embodiments.

FIGS. 3A-3M show an exemplary fabrication process involving depositing a material that is different from the substrate material to form a projection of a capture site in accordance with some embodiments.

FIGS. 4A-4C show an abbreviated exemplary fabrication process in accordance with some embodiments.

FIGS. 5A-5F show various dimensional parameters of a device in accordance with some embodiments.

FIGS. 6A-6C show various mask configurations and resultant etch results.

FIGS. 7A-7B show an exemplary fabrication process for a device comprising an oxide-based device (e.g., feature) layer in accordance with some embodiments.

FIGS. 8A-8C show an abbreviated exemplary fabrication process and a mask in accordance with some embodiments.

FIGS. 9A-9I show an exemplary fabrication process in accordance with some embodiments.

FIGS. 10A-10C provide cross-sections of various well and projection configurations in accordance with some embodiments.

FIGS. 11A-11C show top-down views of various masks in accordance with some embodiments.

### DETAILED DESCRIPTION

### INTRODUCTION

Highly parallel systems, devices and methods for the delivery of payloads to large numbers of cells are used for research and development, diagnostics and therapeutics. For example, stem cell-based therapies benefit from the ability to efficiently transfect large numbers of cells with genetic material or genome editing tools, while maintaining cell viability.

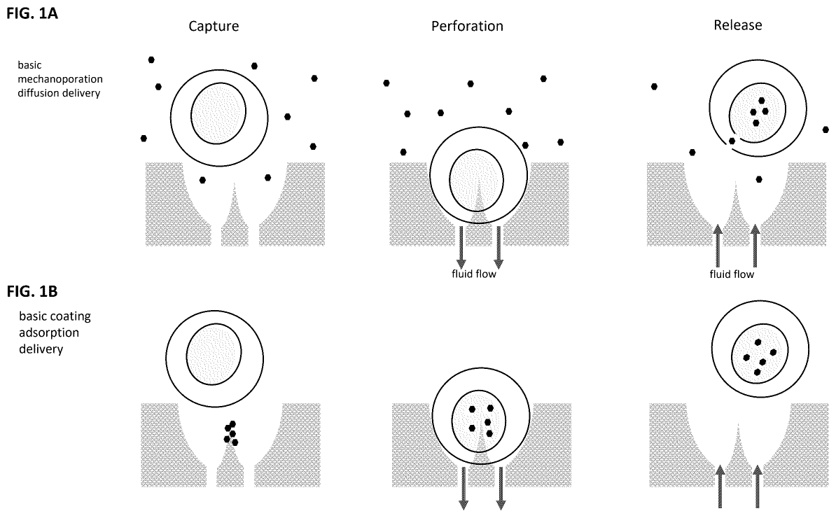

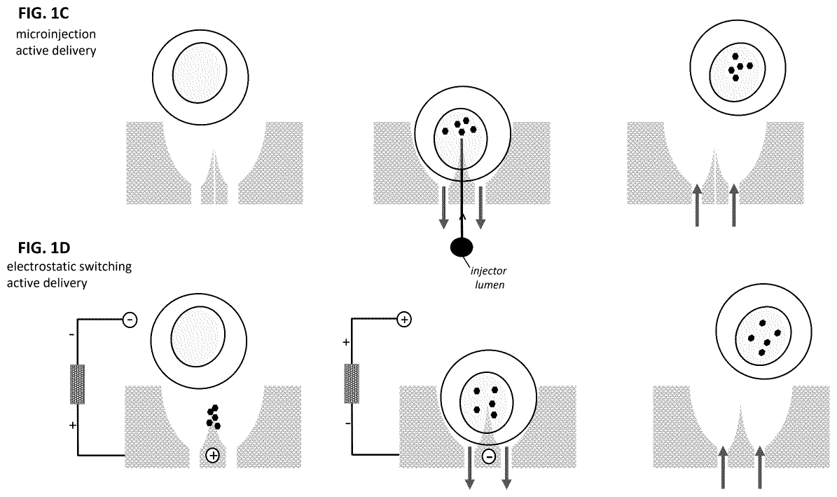

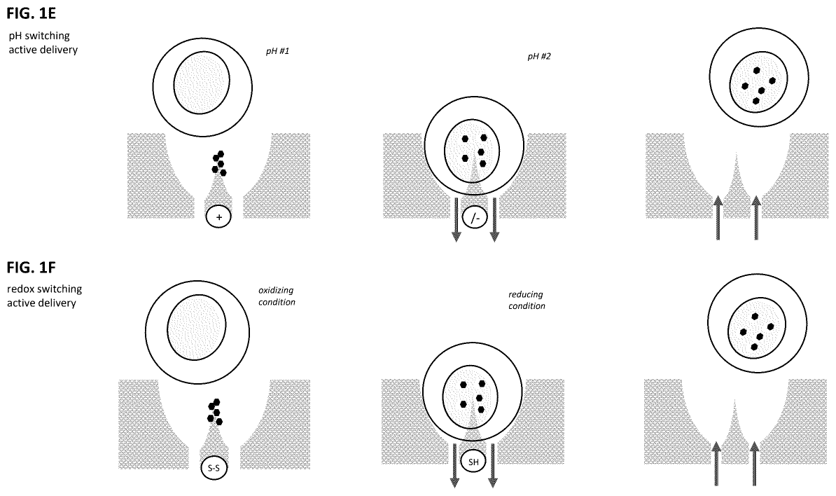

Provided herein are various embodiments of fabrication methods for the fabrication of systems and devices for payload insertion into cells. In certain embodiments, systems, devices, compositions and methods described herein provide for the capture of, perforation (e.g., puncture, poration, or breach) of, release of and payload insertion to cells, as shown in FIGS. 1A-1F. Such embodiments are configured to work with various cell types and may deliver cargos (e.g., payloads) to the nucleus of the cell. In certain aspects, systems, devices, compositions and methods described herein may provide for the transfection of over 250,000 cells/cycle and may transfect up to and above 200 million cells/cycle. In some embodiments, a cycle may be referred to as a process whereby the capture of, perforation of, release of and payload insertion to cells is performed on the device or by the system. In some embodiments, following release of perforated cells, the device or system may be used for additional cycles. As shown in FIGS. 1A-1F, perforation may be utilized to produce a hole in each cell, thus enabling delivery of cargo directly to the interior of the cell and/or nucleus without damaging the cells and maintaining cell viability.

In some embodiments, as shown in FIGS. 1A-1F, operations of various methods described herein may include capture of, perforation of, release of, and insertion of a payload into the cell. In some embodiments, various methods for perforation and insertion (e.g., delivery) of a payload may comprise mechanoporation diffusion delivery of a payload as depicted in FIG. 1A. In some embodiments, various methods for perforation and insertion (e.g., delivery) may comprise adsorption delivery of a payload as depicted in FIG. 1B. In some embodiments, various methods for perforation and insertion (e.g., delivery) may comprise microinjection active delivery of a payload as depicted in FIG. 1C. In some embodiments, various methods for perforation and insertion (e.g., delivery) may comprise electrostatic switching active delivery of a payload as depicted in FIG. 1D. In some embodiments, various methods for perforation and insertion (e.g., delivery) may comprise pH switching active delivery of a payload as depicted in FIG. 1E. In some embodiments, various methods for perforation and insertion (e.g., delivery) may comprise reduction and oxidation (redox) switching active delivery of a payload as depicted in FIG. 1F.

In some embodiments, the operations of a method may take place in an environment filled with the payload. In some embodiments, the operations of a method may take place whereby the payload may be coated, adsorbed or localized onto the surface of the device. In some embodiments, the payload may be specifically coated or absorbed to wells of the device. In some embodiments, the payload may be specifically coated or absorbed to the projection (e.g., needle) of the device. In some embodiments the device surface or portion of the device is treated or coated with compounds or polymers to assist or modulate payload adsorption to the surface of the device. Payload may be nonspecifically absorbed to the device or in other embodiments through specific reversible chemistry or charge interactions. In some embodiments, the payload may be absorbed onto the device using reversible disulfide bond formation and is released into the cell by reduction of the disulfide bond. In some embodiments the payload is absorbed onto the surface of the device in a pH dependent manner and is released to the cells in a pH dependent manner. In some embodiments the payload is absorbed onto the surface of the device in an electrochemical charge dependent manner and is released to the cell by reversing the electrochemical charge applied to the device. In some embodiments the payload is delivered to the cell using microinjection into the cells from the device capture site, including the device wells and projections (e.g., needles). In some embodiments, the payload comprises genetic material. In some embodiments, the genetic material comprises target genes. In some embodiments, the payload comprises gene/genome editing components or tools. In some embodiments, at capture, the cells are distributed into arrayed wells on the device using fluid flow. In some embodiments, fluid flow is controlled in a constant flow manner. In some embodiments, fluid flow is controlled in a constant pressure differential manner. In some embodiments, at perforation (e.g., poration), the cell may be perforated with a single perforation. In certain aspects, at release, the perforated cell may be placed back in the environment for the insertion of the target gene (e.g., “cargo,” “payload” or “genetic material”) into the cell. In some embodiments, at perforation, the insertion of the target gene (e.g., “cargo,” “payload” or “genetic material”) occurs during cell perforation.

In the current art, performance limitations of prevailing gene delivery techniques are preventing full realization of the Cell and Gene Therapy (CGT) industry's therapeutic promise. Currently, two limited capability methods are suitable/currently employed for therapeutic ex vivo gene delivery applications: electroporation and viral vectors.

In the current art, issues involving Viral Vector Transduction include: (1) poor efficiency, particularly for stem and primary human cells; (2) limited versatility, including packaging constraints; (3) limited scalability due to high production cost and long lead times; and (4) safety concerns, including insertional oncogenesis.

In the current art, issues involving electroporation include: (1) poor efficiency, particularly for stem and primary human cells; (2) limited versatility, including challenges with delivery of large cargo to the nucleus; (3) limited scalability, including uniformity challenges; and (4) safety concerns, including functional deficits.

In some embodiments, a nanomechanical method comprising precise (non-stochastic like electroporation) may be used for gene delivery. In some embodiments, mechanoporation allows for: (1) uniform single-site perforation in the cell; (2) nuclear delivery; (3) massive parallelization; (4) rapid cell processing; and (5) simplified operation.

In the current art, delivery of model cargos that require cytosolic localization (e.g., GFP mRNA), and ultimately, a smaller application space. The performance of these competitors is likely lower for delivering large cargos and will struggle to achieve required nuclear localization.

### TERMS AND DEFINITIONS

Unless otherwise defined, all technical terms used herein have the same meaning as commonly understood by one of ordinary skill in the art to which this disclosure belongs.

As used herein, the singular forms “a,” “an,” and “the” include plural references unless the context clearly dictates otherwise. Any reference to “or” herein is intended to encompass “and/or” unless otherwise stated.

As used herein, the term “about” in some cases refers to an amount that is approximately the stated amount.

As used herein, the term “about” refers to an amount that is near the stated amount by 10%, 5%, or 1%, including increments therein.

As used herein, the term “about” in reference to a percentage refers to an amount that is greater or less the stated percentage by 10%, 5%, or 1%, including increments therein.

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

### APPLICATIONS AND REAGENTS

Described herein are various systems, methods and devices for transfecting suspended cells, specifically designed to meet the needs of engineered cell product manufacturing. In some embodiments, systems, devices, and methods are configured for delivery of various cargos to cells. In some embodiments, applications for delivery of various cargos to cells may comprise cellular engineering, gene transfection, genetic deletion, genetic addition, genetic engineering or genetic modification. In some embodiments, methods include deterministic mechanoporation (DMP) of large numbers of cells, each at a single site in their plasma membrane, the methods may allow rapid collection of the cells for subsequent processing. In some embodiments, DMP enables efficient delivery of large-molecule cargos while minimizing damage to the cell, thus allowing achievement of transfection yields that exceed current non-viral transfection techniques. DMP provides a mechanism for addressing roadblocks in the development and manufacture of ex vivo cell therapies based on engineered T cells (e.g., adoptive cancer immunotherapies based on chimeric antigen receptor modified T cells or engineered T cell receptor (TCR) therapies). Applications, systems, devices and methods for delivery of payloads to cells are described in Balas et al. U.S. Pat. No. 9,885,059B2 and Rao et al. US20200289568A1, wherein both references are herein incorporated by reference in their entirety.

In some embodiments, methods may comprise primary human T cell engineering. In some embodiments, methods may utilize model cargos that require nuclear localization (e.g., GFP plasmid). In some embodiments, applications may comprise HDR-mediated large gene insertion.

### Cells

In some embodiments, the systems devices and methods may provide for the mechanoporation of various cell types. In some embodiments, systems, devices and methods may be used with any cell type. In some embodiments, systems, devices and methods may be used with any cell type, wherein the cell type may be prepared as a suspension of single cells. In some embodiments, protoplasts prepared from cells may be used. In some embodiments, the cell preparation may be exposed to isotonic, hypotonic or hypertonic conditions to modulate cell size. In some embodiments, cell origins may comprise human, animal, plant or a combination thereof. In some embodiments, cell types may comprise T cells, Natural Killer Cells (NK Cells), NKT Cells, Monocytes, Myeloid Cells, Hematopoietic Stem Cells, Mesenchymal Stem Cells, Induced Pluripotent Stem Cells (iPSC), Hepatocytes, or Progenitor cells.

### Payload and Surface Coatings

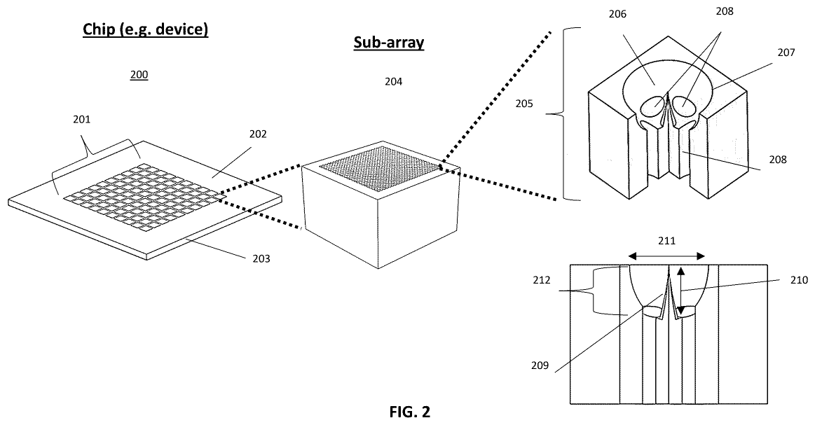

In some embodiments, various payloads may be inserted into a cell. In some embodiments, payloads may comprise chemical compounds (organic or inorganic), proteins, peptides, polymers and nucleic acids. In some embodiments, various payloads may be inserted into a cell as a single agent. In some embodiments, various payloads may be inserted into a cell as mixtures. In some embodiments, the payload DNA may comprise any form of natural or synthetic DNA, including genomic DNA, viral DNA, cDNA, plasmid DNA, dsDNA, ssDNA, oligonucleotides and hybrid sequences or synthetic or semi-synthetic sequences thereof. In some embodiments, the payload RNA may comprise any form of RNA, including viral RNA, RNAi, siRNA, saRNA, mRNA, tRNA, rRNA, microRNA, gRNA, sgRNA and hybrid sequences or synthetic or semi-synthetic sequences thereof. In some embodiments, the payload proteins may comprise enzymes including CRISPR/Cas9 alone or in complex (RNP) with guideRNA or sgRNA complexes for gene editing using CRISPR. In some embodiments, payloads may comprise alternative class enzymes for CRISPR (other than Cas9). In some embodiments, payload proteins may comprise enzymes such as transposases including Sleeping Beauty transposase and Piggy Bac transposase. In some embodiments, the payload may comprise TALENS and other synthetic hybrid molecules such as Cas-CLOVER. In some embodiments, payload proteins may comprise transcription factors natural or synthetic mediating activation or repression of gene expression. In some embodiments, payload proteins or peptides may comprise synthetic or mutated. In some embodiments, the payloads may be delivered in suspension as depicted in FIG. 1A. In some embodiments, the payloads may be present in solutions before, during, and/or after the perforation event. In some embodiments, the payloads may be formulated with condensation agents. In some embodiments these condensation agents may comprise proteins or peptides (protamine, neuphorin, etc.), lipids including cationic lipids (Lipofectamine, dimethyldidodecylammonium bromide, didecyldimethylammonium bromide, dimethyldioctadecylammonium bromide, etc), or polymers including cationic polymers (polyethyleneimine, etc.). In some embodiments, commercial transfection enhancers or transfection agents may be used. In some embodiments, the payload may be injected into the cell through a lumen located within a projection as depicted in FIG. 1C. In some embodiments, the payload may be adsorbed onto the device, well, and/or projection 209, as depicted in FIGS. 1B, 1D-1F and FIG. 2.

In some embodiments, the payload may be adsorbed onto a coating applied to the device, well, and/or projection. In some embodiments the coating may comprise a positively charged molecule, negatively charged molecule, or a molecule of mixed charge. In some embodiments, the coating may be applied as a mixture. In some embodiments, coatings may be applied in series. In some embodiments, the coating comprises a polymer. In some embodiments, the polymer comprises poly-L-lysine, poly-D-lysine, poly-L-arginine, poly-D-arginine, poly-L-ornithine, poly-D-ornithine, poly-L-histidine, poly-D-histidine, poly-L-glutamate, poly-D-glutamate, alginate, hyaluronic acid, polyethyleneimine (PEI), polybrene, chitosan, pullulan, or poly(diallyldimethylammonium chloride). In some embodiments, the coating comprises block copolymers of the polymer with polyethylene glycol (PEG). In some embodiments, the coating comprises block copolymers of the polymer with polyethylene glycol (PEG), such as poly-L-lysine-g-PEG (PLL-g-PEG). In some embodiments, the coating comprises mixtures of the polymer or coblock polymer. In some embodiments, the coating may comprise lipids. In some embodiments the coating comprises cationic lipids such as Lipofectamine, dimethyldidodecylammonium bromide, didecyldimethylammonium bromide, or dimethyldioctadecylammonium bromide. In some embodiments, the coating may comprise polyhistidine containing polymers or mixtures. In some embodiments, the coating may comprise polyhistidine containing polymers or mixtures configured to allow for pH dependent cargo adsorption and release in cell compatible/physiological pH range. In some embodiments, the pH dependent payload (e.g., cargo) adsorption and release in a cell compatible/physiological pH range may be mediated by compounds other than histidine. In some embodiments, the coating comprises a protein. In some embodiments, the coating comprises protamine. In some embodiments, the coating comprises a functionalized silane. In some embodiments, the functionalized silane comprises an amino terminated silane. In some embodiments, the amino terminated silane comprises 3-Aminopropyltriethoxysilane (APTES), Trimethoxysilylpropyldiethylenetriamin (DETA), N-(2-aminoethyl)-3-aminopropyltrimethoxysilane (EDA), m,p-(aminoethyl-aminomethyl)phenethyltrimethoxysilane (PEDA), or a silane-PEG-amine. In some embodiments, the coating comprises a sulfhydryl terminated silanes. In some embodiments, the sulfhydryl terminated silane comprises 3-(mercaptopropyl)trimethoxysilane (MPTES/MTS) or mercaptomethyldimethylethoxysilane (MDS). In some embodiments, the coating comprises polyethyleneglycol (PEG) silanes. In some embodiments, the PEG-silanes comprise Methoxy-PEG-triethoxysilane (m-PEG-silane). In some embodiments, the coating comprises an inorganic material. In some embodiments, the coating comprises a metal. In some embodiments, the metal may comprise gold, zinc, or nickel. In some embodiments the coating may comprise a nitride material. In some embodiments, the coating may comprise carbon. In some embodiments, the coating may be applied by bulk adsorption, sputtering, evaporation, spotting, printing, or electrodeposition methods.

### Other Reagents

In some embodiments, system, devices, kits, or methods may comprise a buffer. In some embodiments, the buffer may comprise Phosphate Buffered Saline (PBS), Dulbecco's Phosphate Buffered Saline (DPBS), Hanks Balanced Salt Solution (HBSS), or HEPES.

In some embodiments, system, devices, kits, or methods may comprise media. In some embodiments, the media may comprise OptiMEM (Gibco), RPMI, X-Vivo15 (Lonza), X-Vivo20 (Lonza), CTS Optimizer T Cell Expansion SFM (Thermo), CTS AIM V Medium (Thermo), CTS KnockOut SR XenoFree Medium (Thermo), or CTS StemPro MSC SFM (Thermo).

In some embodiments, system, devices, kits, or methods may comprise serum and serum replacements. In some embodiments, the serum and serum replacements may comprise FBS, FCS, Human Serum, BSA, HSA, or CTS Immune Cell Serum Replacement (Thermo).

In some embodiments, system, devices, kits, or methods may comprise a surfactant. In some embodiments, the surfactant may comprise F-68 (Poloxamer 188, Kolliphor P 188, Lutrol F68, Pluronic F68, Synperonic F68). In some embodiments, the surfactant may comprise F-108 (Poloxamer 338, Pluronic F108, Synperonic F108). In some embodiments, system, devices, kits, or methods may comprise and antioxidant, anti-apoptotic or cell protective components. In some embodiments the antioxidant, anti-apoptotic or cell protective component may comprise vitamin E, TROLOX, dexamethasone, RHO/ROCK inhibitors (e.g., Y27632, thiazovivan), pyruvate, ascorbate, or n-acetylcysteine (NAC). In some embodiments, system, devices, kits, or methods may comprise a transfection enhancer. In some embodiments, the transfection enhancer may inhibit or modulate cytosolic nucleic acid sensors such as Absent in Melanoma 2 (AIM2) and cyclic GMP-AMP synthase (cGAS). In some embodiments, the transfection enhancer may inhibit or modulate toll-like receptor (TLR) signaling pathways such as TLR7 and/or TLR9. In some embodiments, these transfection enhancers may comprise NATE (InvivoGen), K4 Multiplier (Biontex), or Trans-Booster (RJH Bioscience), chloroquine, E4664 or AT791. In some embodiments, system, devices, kits, or methods may comprise compounds or treatments that increase the size of nuclear pores in the cell. In some embodiments the compounds or treatments that increase the size of nuclear pores in the cell may comprise dexamethasone.

### DEVICE

### Introduction

Described here are various embodiments of a device configured for inserting a payload into a cell. FIG. 2 shows an example system construction. In some embodiments, the device 200 comprises an array of sub-arrays 201, a surface 202, a substrate 203, or a combination thereof. In some embodiments, the array of sub-arrays 201 may be referred to as an array or full array. In some embodiments, sub-arrays 201 may comprise a plurality of capture sites 205. In some embodiments, the capture site 205 comprises a well 206, a well boundary shape 207, a port 208, a projection 209 or a combination thereof. In some embodiments, each capture site of the device may be configured for capture of a single cell. Shown in FIG. 2 is a non-limiting example of a single sub-array 204 comprising approximately 2372 capture sites. As an example, a full device 200, comprising approximately 100 sub-arrays and approximately 237,200 capture sites are shown in FIG. 2. In some embodiments, a single sub-array 201 may comprise approximately 10,000 capture sites 205. In some examples, a full device 200 comprising an array of sub-arrays 201, the array of sub-arrays may comprise approximately 100 sub-arrays 204 and approximately 1,000,000 capture sites 205.

### Array of Sub-Arrays

In some embodiments, the array of sub-arrays 201 comprises at least 1 sub-array 204. In some embodiments, the array of sub-arrays 201 comprise about 100 sub-arrays 204. In some embodiments, the array of sub-arrays 201 comprise sub-arrays 204 arranged in a linear (e.g., square packed) pattern. In some embodiments, the device 200 comprises one array of sub-arrays 201. In some embodiments, device 200 comprises at least one array of sub-arrays 201. In some embodiments the device 200 comprises two or more array of sub-arrays 201 arranged on one or more surfaces 202 of the device 200.

### Sub-Arrays

In some embodiments, the device 200 comprises at least one sub-array 204. In some embodiments, the device 200 comprises from about 1 sub-array 204 to about 140,000 sub-arrays 204. In some embodiments, the device comprises an array of sub-arrays 201, wherein the sub-arrays 204 are arranged in an ordered fashion. In some embodiments, the device 200 comprises an array of sub-arrays 201, wherein the sub-arrays 204 are arranged in a random fashion. In some embodiments, the device 200 comprises an array of sub-arrays 201, wherein the sub-arrays 204 are arranged in a linear fashion. In some embodiments, the sub-array 204 comprises a capture site 205. In some embodiments, the sub-array 204 comprises at least one capture site 205. In some embodiments, the sub-array 204 comprise from one capture site 205 and about 2,000,000,000 capture sites 205. In some embodiments, the sub-array 204 comprises about 2372 capture sites 205. In some embodiments, the capture sites 205 are arranged in the sub-array 204 in a linear (e.g., square) pattern. In some embodiments, the capture sites 205 are arranged in a diamond pattern (e.g., face centered, hexagonal or cubic centered) pattern. In some embodiments, the capture sites 205 are arranged in a random pattern. In some embodiments, the capture sites 205 of the sub-array 204 are arranged in a hexagonal pattern.

### Substrate

Described herein are various devices 200 comprising substrates 203. In some embodiments, the substrate 203 may comprise silicon, semiconductors, silicon, silicon dioxide. quartz, doped silicon, glass, plastics, ceramics, and metals or any combination thereof.

In some embodiments, the substrate may comprise silicon-on-insulator (SOI) substrates. In some embodiments, the insulator may comprise silicon oxide. In some embodiments, the insulator layer may be referred to as a buried oxide (BOX) layer. In some embodiments, the SOI substrates may comprise a device layer, an insulator layer, a handle layer, or a combination thereof.

In some embodiments, the handle layer thickness is about 300 microns to about 950 microns. In some embodiments, the handle layer thickness is at least about 300 microns.

In some embodiments, the device layer thickness is about 5 microns to about 100 microns. In some embodiments, the device layer thickness is at least about 5 microns. In some embodiments, the device layer thickness is about 30 microns.

In some embodiments, the substrate is a crystalline silicon wafer. In some embodiments the crystalline silicon wafer comprises a crystal orientation. In some embodiments, the crystal orientation comprises of 100, 111, or 110. In some embodiments, substrate materials comprise of semiconductors, silicon, doped silicon, glass, plastics, ceramics, and metals or any combination thereof. In some instances, silicon may be doped. In some embodiments, substrate materials may be polycrystalline or amorphous. In some embodiments, substrate material size and dimensions determine the ease of manufacturing.

Described herein are various embodiments of methods for the fabrication of devices. Also described are various embodiments of methods for the fabrication of a projection array (e.g., microneedle array). In some embodiments, a cross sectional view of various parts of the process of forming a capture site 205 comprising a projection (e.g., microneedle) 209, a well 206, and ports 208 as depicted in FIG. 2. In some embodiments, the substrate contains a masking layer, a feature (well/projection/ports) layer, and a structural layer. The layers may comprise single or multiple patterning layers, masking layers, feature layers, and/or structural layers. In some embodiments, the layers may be silicon in single crystal, polycrystalline, or amorphous structure. In some embodiments the layers may comprise of silicon nitride, silicon dioxide, buried oxide layer, silicon carbide, gallium arsenide, gallium phosphide, indium phosphide, indium arsenide, a semiconductor alloy or combination thereof. In certain aspects, the oxide layer can be formed by plasma, thermal oxidation, or chemical vapor deposition (CVD). The patterning layer thickness can be from about 0.1 to about 10 microns. The masking layer thickness can be from about 0.1 to about 100 microns. The feature layer thickness can be from about 0.1 to about 100 microns. The structural layer thickness can be from about 0.1 to about 1000 microns.

Described herein are various embodiments of methods for the fabrication of devices. Also described are various embodiments of methods for the fabrication of a projection (e.g., microneedle) array. In some embodiments, the substrate can have single or multiple patterning layers, masking layers, an oxide layers (e.g., oxide feature layer), and structural layers. In some embodiments the layers can be silicon in single crystal, polycrystalline, or amorphous structure. In some other embodiments the layers can be made of silicon nitride, silicon dioxide, buried oxide, silicon carbide, gallium arsenide, gallium phosphide, indium phosphide, indium arsenide, a semiconductor alloy or combination thereof. In some other embodiments there can be more than one masking, oxide, and structural layers. The oxide layer may be formed by methods comprising atomic layer deposition (ALD), plasma (e.g., plasma-enhanced chemical vapor deposition (PECVD), thermal oxidation, or chemical vapor deposition (CVD). The masking layer thickness can be 0.1 to 10 microns. The oxide layer thickness can be 0.1 to 100 microns. The structural layer thickness can be 0.1 to 1000 microns.

### Capture Sites

Described herein are various devices 200, comprising: a substrate 203 comprising: a surface 202; and a capture site 205, the capture site comprising: a well 206 having a well boundary shape (e.g., circle) 207 at the surface 202 and having a well depth 212 and a well shape diameter 211, the well configured to support a cell; and a projection 209 extending from the bottom of the well toward the surface 202 of the substrate 203, the projection 209 comprising a projection length 210, and wherein the projection length 210 and the well depth 212 comprise a projection length-well depth ratio. In some embodiments the capture site 205 comprises a port 208. In some embodiments, the capture site 205 comprises a plurality of ports 208, as shown in FIG. 2. In some embodiments, the capture site is configured for the capture of, perforation (e.g., poration) of and release of a cell. In some embodiments, the well boundary shape 207 comprises a well boundary perimeter.

### Well

In some embodiments, the capture site 205 comprises a well 206. In some embodiments, the well 206 is configured for the capture of a cell as shown in FIGS. 1A-1F. In some embodiments, the well boundary shape 207 comprises a circle, ellipse, square, rectangle, triangle, or any combination thereof. In some embodiments, the well boundary shape 207 comprises a well boundary shape area. In some embodiments, the well boundary shape 207 comprises a circle. In some embodiments, the well boundary shape 207 comprises a well diameter. In some embodiments, the well boundary shape 207 comprises a perimeter. In some embodiments, the well boundary shape diameter is about 100 microns and about 1 micron. In some embodiments, the well boundary shape diameter is about 10 microns.

In some embodiments, the well depth 212 is about 50 microns to about 0.5 microns. In some embodiments, the well depth is about 7.5 microns to about 5 microns. In some embodiments, the well depth is about 5 microns.

In some embodiments, the well perimeter is about 160 microns to about 1.5 microns.

### Projection

In some embodiments, the capture site 205 comprises a projection 209. In some embodiments, the capture site 205 comprises one projection 209. In some embodiments, the capture site 205 comprises a plurality of projections 209. In some embodiments, the plurality of projections comprises 2 projections to about 100 projections. In some embodiments, the projection 209 is configured to perforate the cell, forming a perforation (e.g., pore) in the cell membrane and/or cell wall as shown in FIGS. 1A-1F.

In some embodiments, the projection 209, comprises a projection length 210. In some embodiments, the projection length 210 is about 50 microns to about 0.5 microns. In some embodiments, the projection length is about 6 microns to about 2 microns. In some embodiments, the projection length is about 3.5 microns. In some embodiments, the projection may be referred to as a needle. In some embodiments, the projection length may be configured for flow focusing above the projection tip.

In some embodiments, the projection length may be engineered to accommodate a target cell to be captured. In some embodiments, the projection length is configured for a projection length to cell diameter ratio. In some embodiments, the projection length to cell diameter ratio may be determined by the diameter of the cell chosen, specifically to enter the nucleus but not go thru the entire cell.

In some embodiments, the projection comprises a projection tip. In some embodiments, the projection tip diameter is measured at a distance of 50 nm from the absolute tip (e.g., projection tip end). In some embodiments, the projection comprises a projection base diameter. In some embodiments, a projection comprises a projection tip diameter to projection base diameter ratio. In some embodiments, the projection tip diameter is about 100 nm. In some embodiments, the projection base diameter is about 2000 nm.

In some embodiments, the projection tip diameter to projection base diameter is about 100:2000. In some embodiments, the projection tip diameter to projection base diameter is about 1:20. In some embodiments, the projection tip diameter to projection base diameter is about at most 1:20. In some embodiments, the projection tip diameter to projection base diameter is about at least 1:20.

### Projection Length—Well Depth Ratio

Described herein are various devices 200, comprising: a substrate 203 comprising: a surface 202; and a capture site 205, the capture site 205 comprising: a well 206 having a well boundary shape 207 at the surface 202 and having a well depth 212, the well 206 configured to support a cell; and a projection 209 extending from the bottom of the well toward the surface 202 of the substrate 203, the projection 209 comprising a projection length 210, and wherein the projection length 210 and the well depth 212 comprise a projection length-well depth ratio. In some embodiments, the projection length-well depth ratio is from about 10% to about 100%. In some embodiments, the projection length-well depth ratio is from about 40% to about 60%. In some embodiments, the projection length-well depth ratio is about 50%.

### Ports

In some embodiments, capture site 205 comprises a port 208. In some embodiments, the port 208 comprises an aspiration via. In some embodiments, the aspiration via is connected a pump. In some embodiments, the capture site 205 is configured for the capture of, perforation of, and release of, a cell, as shown in FIGS. 1A-1F. In some embodiments, the port 208 comprises a port boundary shape. In some embodiments, the port boundary shape comprises a circle, ellipse, square, rectangle, triangle, arc via or a partial arc via (e.g., arc). In some embodiments, the port 208 comprises a port boundary shape area. In some embodiments, the port boundary shape comprises a long axis. In some embodiments, the port comprises a plurality of ports. In some embodiments, the plurality of ports comprises about 1 port to about 100 ports. In some embodiments, the port arrangement comprises of circular, elliptical, square, rectangular, triangular, diamond or hexagonal. In some embodiments, the port 208 comprises a lumen. In some embodiments, the projection 209 comprises the lumen. In some embodiments, the plurality of ports comprises a lumen and/or an aspiration via. In some embodiments, the capture site 205 comprises a plurality of ports. In some embodiments, the port boundary shape area comprises a port boundary shape area from about 1 micron squared to about 100 microns squared. In some embodiments, the aspiration via arrangement and shape are configured for uniformity of velocity magnitude across the channel length, inside and above the well.

In some embodiments, the port comprises a port channel, the port channel comprising a port channel length. In some embodiments the port channel length may be about 5 microns to about 100 microns. In some embodiments, the port channel length is greater than 3 microns. In some embodiments, the port channel length is greater than 5 microns. In some embodiments, the port length is greater than 10 microns. In some embodiments, the port channel length is greater than 20 microns. In some embodiments, the port length is 22.5 microns. In some embodiments, the port channel length is 24 microns. In some embodiments, the port length is 25 microns.

In an aspect, provided is a device 200 comprising: a surface 202; and a capture site 205, the capture site comprising: a well 206 having a well boundary shape at the surface and having a well depth, the well configured to support a cell; and a projection 209 extending from the bottom of the well toward the surface of a substrate 203, and wherein the projection does not move relative to the substrate as depicted in FIG. 2.

In an aspect, provided device 200 comprising: a surface 202; and a capture site 205, the capture site comprising: a well 206 having a well boundary shape at the surface and having a well depth, the well configured to support a cell; and a projection 209 extending from the bottom of the well toward the surface of a substrate 203, the projection comprising a projection length, and wherein the substrate material is different than the feature material as depicted in FIG. 2.

In some embodiments, the substrate contains a masking layer, a device layer (e.g., feature layer), and a structural layer. In some embodiments, the device layer may be the portion of the substrate wherein wells, ports or projections may be fabricated. In some embodiments, the layers can have single or multiple patterning layers, masking layers, feature layers, and/or structural layers. In some embodiments, the layers can be silicon in single crystal, polycrystalline, or amorphous structure. In some embodiments the layers can be made of silicon nitride, silicon dioxide, buried oxide layer, silicon carbide, gallium arsenide, gallium phosphide, indium phosphide, indium arsenide, a semiconductor alloy or combination thereof. In some embodiments, the oxide layer can be formed by methods comprising plasma, thermal oxidation, or chemical vapor deposition (CVD). The patterning layer thickness can be from about 0.1 to about 10 microns. The masking layer thickness can be from about 0.1 to about 100 microns. The feature layer thickness can be from about 0.1 to about 100 microns. The structural layer thickness can be from about 0.1 to about 1000 microns.

In some embodiments a substrate 300 may comprise a device layer, an insulator layer, a handle layer, a mask (e.g., masking layer) or a combination thereof. In some embodiments, the mask may comprise an oxide layer. In some embodiments, the masking layer may comprise a silicon dioxide layer. In some embodiments the masking layer may comprise a 1 micron thick silicon dioxide layer.

In some embodiments, the substrate may comprise a handle layer and a device layer, wherein the device layer may comprise the insulator layer. In some embodiments, wherein the device layer may comprise the insulator layer, the device layer may comprise an oxide material. In such embodiments, the device layer may comprise silicon dioxide.

In some examples, the precision and accuracy of the projection length across the surface of the substrate (e.g., device 200) may allow for consistent gene transfection processing as depicted in FIGS. 1A-1F. In some examples, the precision and accuracy of the projection length across the surface of the substrate is represented by the coefficient of variance (CV or % CV) of the projection length to well-depth ratio. In some examples, the precision and accuracy of the projection length across the surface of the substrate is represented by the coefficient of variance (CV or % CV) of the recess gap.

In some embodiments, the % CV of the projection length to well depth ratio for the device may be about 0.1% to about 25%. In some embodiments, the % CV of the projection length to well depth ratio for the device may be about 1% to about 10%. In some embodiments, the % CV of the projection length to well depth ratio for the device may be less than about 1%.

In some embodiments, the % CV of the recess gap for the device may be about 0.1% to about 25%. In some embodiments, the % CV of the recess gap for the device may be about 1% to about 10%. In some embodiments, the % CV of the gap for the device may be less than about 1%.

### METHODS

### Patterning

In some embodiments of the method for fabrication, a mask may be used to expose the substrate to various etchants at select locations. In some embodiments a hard mask (e.g., oxide) may be used. In some embodiments, a soft mask (e.g., polymer) may be used. In some embodiments, a soft mask may be formed by photolithography. In some embodiments a hard mask may be formed by a combination of photolithography followed by etching of the preliminary layer comprising the mask material (e.g., masking layer) to expose regions of the substrate (e.g., surfaces of the device layer or handle layer). In such embodiments, wherein a hard mask may be formed, the photolithographically produced soft mask may be removed, yielding a hard mask. In some embodiments, the soft mask (e.g., patterning layer) may comprise a thickness of about 0.1 to about 10 microns. In some embodiments, the masking layer may comprise silicon dioxide.

In some embodiments, patterning may comprise alignment of the pattern of features of a mask on the back side of the wafer with the pattern of features of a mask on the on the front side of the wafer.

### Chemical Mechanical Polishing (CMP)

In some embodiments, chemical mechanical polishing (e.g., CMP) may be used for planarizing or thinning substrates.

### Dry Etching/Milling

In some embodiments, the dry etching may be referred to as plasma etching. In some embodiments, dry etching may comprise anisotropic (e.g., in one direction) dry etching. In some embodiments, dry etching may be configured to produce anisotropic trenches in silicon. In some embodiments, dry etching may be configured to produce anisotropic trenches in an oxide. In some embodiments, dry etching may be configured to produce anisotropic trenches in silicon oxide. In some embodiments, the dry etching may be referred to as milling. In some embodiments, the dry etching may comprise argon sputter etching, ion milling, plasma etching, or reactive ion etching. In some embodiments, the substrate may be oriented perpendicular to the direction of the etchant (e.g., argon ions). In some embodiments, the substrate may be oriented at an oblique angle relative to the etchant (e.g., argon ions). In some embodiments, the substrate may be rotating during the dry etching. In some embodiments, the substrate may be oriented at an oblique angle relative to the etchant and the substrate may be rotating.

### Wet Etching

In some embodiments, wet etching may be used. In some embodiments, wet etching may comprise isotropic (e.g., in all directions) wet etching. In some embodiments, wet etching may be configured to isotropically etch silicon. In some embodiments, the isotropic wet etching of silicon may comprise an etchant comprising sodium hydroxide.

### Depositions

In some embodiments, the depositing may comprise atomic layer deposition (ALD), sputtering, physical vapor deposition (PVD), or other deposition processes. In some embodiments, the feature material (e.g., fill or deposited material) may comprise silicon dioxide, aluminum dioxide, hafnium dioxide, titanium dioxide, chrome oxide, chromium oxide, zirconium oxide, zinc oxide, silicon nitride, tungsten, platinum, boron nitride, and titanium nitride.

### Method of Manufacture of the Device

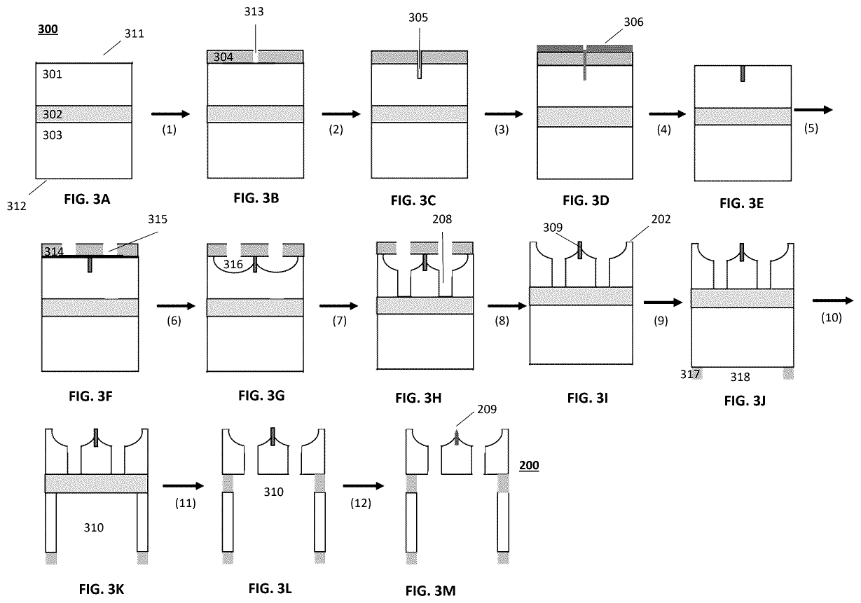

Described herein are various embodiments of a method for fabricating a device 200 as depicted in FIGS. 3A-3M. In some embodiments, a substrate 300 may be obtained or provided, wherein the substrate may comprise a device layer (e.g., feature layer) 301, an insulator layer (e.g., buried oxide layer or BOX layer) 302, a handle layer (e.g., structural layer) 303, a front side 311 and a back side 312 as depicted in FIG. 3A.

In some embodiments, the method may comprise an operation (e.g., operation (1)) comprising forming a mask 304 on the front side 311 of the substrate 300 of FIG. 3A, yielding the substrate of FIG. 3B. In such an embodiment, the mask 304 may comprise an opening 313 exposing the device layer to etchants.

In some embodiments, the method may comprise etching. In some embodiments, the method may comprise an operation (e.g., operation (2)) comprising anisotropic dry etching of the substrate 300 as depicted in FIG. 3B, yielding the substrate comprising a feature region (e.g., cavity) 305 as depicted in FIG. 3C.

In some embodiments, the method may comprise depositing a material (e.g., feature material). In some embodiments, the method may comprise an operation (e.g., operation (3)) comprising depositing a material through an opening of the mask 304 of substrate 300 as depicted in FIG. 3C, yielding the substrate as depicted in FIG. 3D comprising a deposited material 306.

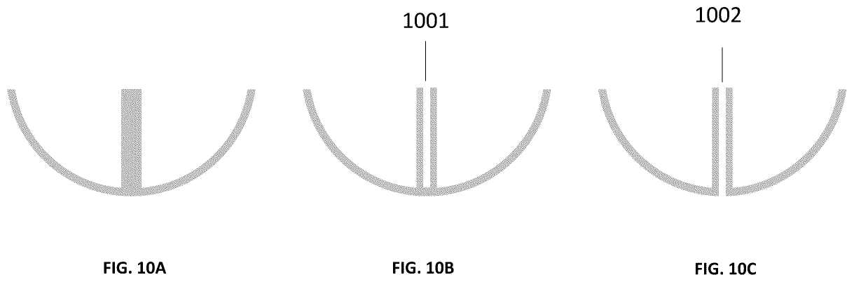

In some embodiments, a hollow projection (e.g., needle) can be formed by depositing material, at a fraction of the projection diameter (e.g., within a fraction of the projection's top-down cross-sectional area). In some embodiments, a hollow projection (e.g., needle) can be formed by depositing material, at a fraction of the projection diameter, wherein depositing will create a conformal film that adheres to the sidewalls and does not fill the center. A side cross-section of a hollow projection can be seen in FIG. 10B. A side cross-section of a solid projection is depicted in FIG. 10A. In some embodiments, a hollow projection can be formed by depositing material, located at a fraction of the projection diameter, wherein depositing will create a conformal film that adheres to the sidewalls and does not fill the center, wherein the depositing comprises atomic layer deposition (ALD). In some embodiments, the surface at the bottom of the hollow projection (e.g., needle) can be removed by a directional plasma etch, forming a hollow projection with an opening at the bottom 1002 of the well as depicted in FIG. 10C.

In some embodiments, a hollow projection 1001 can be formed by adding a mask to cover a fraction of the projection (e.g., needle) center. In some embodiments, a hollow projection 1001 can be formed by adding a mask to cover a fraction of the projection (e.g., needle) center and depositing material around the inner circle creating a ring using ALD.

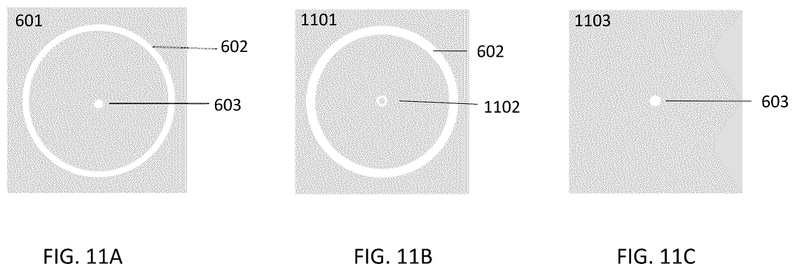

In some embodiments, the method may comprise an operation comprising adding a mask 1103 as depicted in FIG. 11C to cover outside the perimeter of the well and expose the projection, while dry etching to shorten the projection height. FIG. 11A shows the mask 601 in accordance with some embodiments. FIG. 11B shows a mask 1101 comprising an inner annular opening configured to produce a hollow projection.

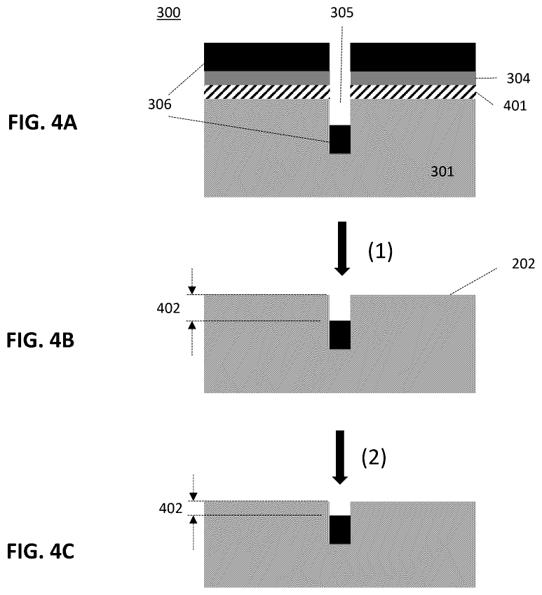

In some embodiments, the method may comprise an operation (e.g., operation (4)) comprising removing the deposited material 306 and the feature mask 304 all in the one operation, yielding the substrate 300 as depicted in FIG. 3E. In some embodiments, the removing may comprise lifting-off the feature mask and deposited material as depicted in operations (1) and (2) of FIGS. 4A-4C. In some embodiments, the lifting-off comprises obtaining or providing a substrate as depicted in FIG. 4A or equivalently, as depicted in FIG. 3D. As depicted in FIG. 4A, the substrate may comprise a lift-off layer 401. In some embodiments, the lifting-off comprises the operation (1) of FIGS. 4A-4C, yielding the substrate of FIG. 4B. In some embodiments, the distance between the front side device surface 202 and the top surface of the deposited material in the cavity 305 is referred to as the recess gap 402 as depicted in FIGS. 4B and 4C. In some embodiments, the operation (1) of FIGS. 4A-4C may comprise dissolving the lift-off layer 401, thereby lifting-off (e.g., removing) the mask 304 and the excess deposited material 306 residing on top of the mask. Since both the substrate material and the deposited material are not soluble in the solvent used in the lift off process (e.g., operation (1)) of FIGS. 4A-4C, then the deposited feature material 306 in the cavity 305 may be retained in accordance with some embodiments. In some embodiments, lifting-off may not require the need to remove the mask 304 by etching or CMP, thereby allowing for the original high-quality factory surface of the substrate (e.g., wafer) to be maintained. However, if desired further processing to reduce the height of the front side 311 may be implemented by etching, CMP or other methods as depicted by operation (2) of FIGS. 4A-4C. In some embodiments, the distance between the finalized device surface 202 and the top surface of the deposited material 306 may be referred to as the recess gap 402 as depicted in FIG. 4C.

The deposition process (e.g., operation (1) of FIGS. 4A-4C) of the lift-off process may allow for precise and accurate control of the thickness of the deposited material 306. The deposition process (e.g., operation (1) of FIGS. 4A-4C) of the lift-off process may allow for precise and accurate control of the thickness of the deposited material across the area of the front side of the substrate. The precision and accuracy of the thickness of the deposited material may result in the precision and accuracy of the projection length. In some embodiments, the precision and accuracy of the thickness of the deposited material may result in the precision and accuracy each the features of each capture site of a plurality of capture sites of the device. In some examples, the precision and accuracy of the projection length across the surface of the substrate (e.g., device 200) may allow for precise and accurate gene transfection processing as depicted in FIGS. 1A-1F. In some examples, the precision and accuracy of the projection length across the surface of the substrate is represented by the coefficient of variance (CV or % CV) of the projection length to well-depth ratio. In some examples, the precision and accuracy of the projection length across the surface of the substrate is represented by the coefficient of variance (CV or % CV) of the recess gap.

In some embodiments, the % CV of the projection length to well depth ratio for the device may be about 0.1% to about 25%. In some embodiments, the % CV of the projection length to well depth ratio for the device may be about 1% to about 10%. In some embodiments, the % CV of the projection length to well depth ratio for the device may be less than about 1%.

In some embodiments, the % CV of the recess gap for the device may be about 0.1% to about 25%. In some embodiments, the % CV of the recess gap for the device may be about 1% to about 10%. In some embodiments, the % CV of the gap for the device may be less than about 1%.

In some embodiments, the method may comprise an operation (e.g., operation (5)) comprising forming a mask 314 on the front side 311 of the substrate 300 of FIG. 3E, yielding the substrate of FIG. 3F. In some embodiments, the mask may comprise openings 315 exposing the device layer to etchants.

In some embodiments, the method may comprise etching the front side 311 of the substrate to form a well. In some embodiments, the method may comprise an operation (e.g., operation (6)) comprising isotropically wet etching of the front side 311 of the substrate 300 as depicted in FIG. 3F, yielding the substrate as depicted in FIG. 3G. As depicted in FIG. 3G, each opening 315 of the mask 314 allows for etchant to form a well 316 in the substrate.

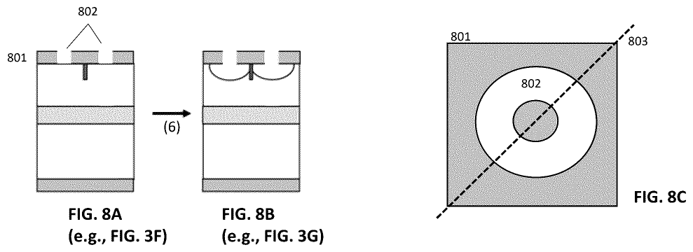

In some embodiments, a single circular trench 804 may be etched due to an annular opening 802 of mask 801 as depicted in FIG. 8C. The dashed line 803 of FIG. 8C indicates the cross section as viewed in FIGS. 8A, 8B and 3F-3H.

In some embodiments, wherein the mask 801 comprising a single circular opening 802 as depicted in FIGS. 8A-8C, may be removed and a port mask (e.g., aspiration via mask) comprising four openings per well may be formed on the surface of the substrate. In such embodiments, the port mask (e.g., aspiration via mask) the four openings configured to allow for four ports (e.g., aspiration vias, aspiration via channels) to be etched within each well boundary perimeter.

In some embodiments, the port mask (e.g., aspiration via mask) comprising four circular openings per capture site may be used for both the formation of the well and formation of the aspiration vias (e.g., operations (6) and (7) of FIGS. 3A-3M). In such an embodiment, the well boundary perimeter comprises a clover shape due to each of the four isotropically etched wells overlapping during the etch to form the composite-clover shape. In some embodiments, the method may comprise anisotropically dry etching through the opening 315 of mask 314, after formation of the well 316 (e.g., operation (7)) to form the aspiration via 208 as depicted in FIG. 3H.

In some embodiments, the method may comprise removing the mask 314 by etching (e.g., operation (8)) yielding the substrate as depicted in FIG. 3I. In some embodiments, the mask 314 may be an oxide-based hard mask. In some embodiments, the oxide-based hard mask may be removed by dry etching utilizing an etchant configured to etch silicon dioxide at a faster rate versus silicon, thereby allowing the silicon structure to be left in place, while the silicon dioxide is removed. Upon removal of the mask 314, a blunt projection 309 may be present as depicted in FIG. 3I.

In some embodiments, the method may comprise an operation (e.g., operation (9)) comprising forming a mask 317 on the back side surface 312 of the substrate 300 of FIG. 3J. In some embodiments, the mask 317 may comprise an opening 318 exposing the handle layer (e.g., structural layer) 303. In such embodiments, the opening 318 may be aligned to a sub-array 204 on the front side 311 of the substrate. Subarrays 204, of certain aspects, can be seen in FIG. 2.

In some embodiments, the method may comprise and operation (e.g., operation (10)) comprising forming (e.g., dry etching) a back side sub-array trench 310 into the handle layer 303 yielding the substrate 300 as depicted in FIG. 3K. In some embodiments, the back side sub-array trench may be referred to as the sub-array trench.

In some embodiments, the method may comprise and operation (e.g., operation (11)) comprising etching the insulator layer (e.g., BOX layer) 302 to connect the aspiration via channels 208 on the front side of the substrate to the back side sub-array trench 310 as depicted in FIG. 3L. In some embodiments, the etching to remove the BOX layer may comprise dry etching.

In some embodiments, method may comprise sharpening the blunt projection 309 (e.g., operation (12)) yielding the substrate comprising a projection 209 as depicted in FIG. 3M. In some embodiments, the sharpening may comprise ion milling (e.g., Argon sputter etching). In examples, sharpening may comprise rotating the substrate while ion milling the substrate. In some embodiments, the ion milling may comprise Argon sputter etching, wherein the Argon ions are directed to the surface of substrate at an oblique angle. In some embodiments, the ion milling may comprise Argon sputter etching, wherein the Argon ions are directed to the surface of substrate at an oblique angle while the substrate may be rotating. In some embodiments, the oblique angle may be about 45 degrees. In some embodiments, operation (12) of FIGS. 3A-3M may be referred to as projection sharpening. In some embodiments, sharpening may also round the edges of the well (e.g., composite well). In such an embodiments, after sharpening the blunt projection 309, the blunt projection can be referred to as the sharpened projection, or projection 209 as depicted in FIG. 3M.

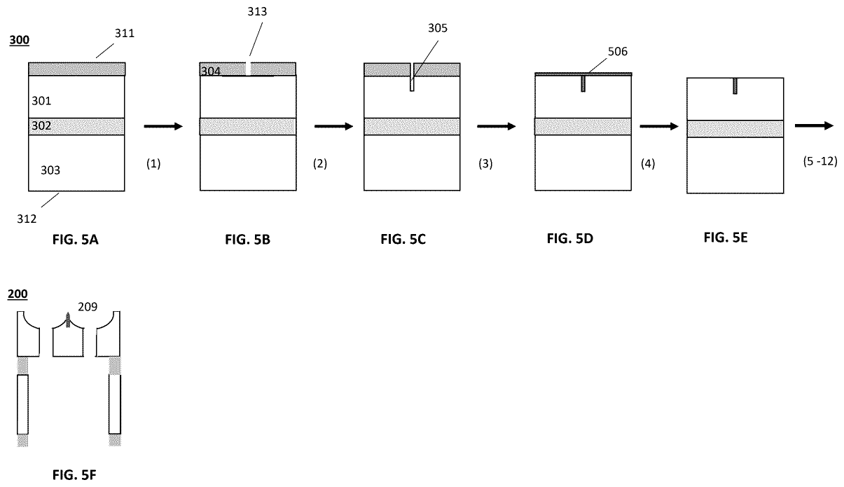

FIGS. 5A-5F show an abbreviated alternative method for fabricating the device 200 in accordance with some embodiments. In some embodiments, a substrate 300 may be obtained or provided, wherein the substrate may comprise a device layer (e.g., feature layer) 301, an insulator layer (e.g., buried oxide layer or BOX layer) 302, a handle layer (e.g., structural layer) 303, a front side 311 and a back side 312 as depicted in FIG. 5A.

In some embodiments, the method may comprise an operation (e.g., operation (1)) comprising forming a mask 304 on the front side 311 of the substrate 300 of FIG. 5A, yielding the substrate 300 of FIG. 5B. In such an embodiment, the mask 304 may comprise an opening 313 exposing the device layer to etchants.

In some embodiments, the method may comprise etching. In some embodiments, the method may comprise an operation (e.g., operation (2)) comprising anisotropic dry etching of the substrate 300 as depicted in FIG. 5B, yielding the substrate 300 comprising a feature region (e.g., cavity) 305 as depicted in FIG. 5C. In some embodiments, the etching may comprise dry etching.

In some embodiments, the method may comprise an operation (e.g., operation (3)) comprising removing the mask 304 and depositing a feature material 506 onto the front side 311 of the substrate yielding the substrate as depicted in FIG. 5D. In some embodiments, the mask 304 may be a hard mask. In some embodiments, the mask may be an oxide-based mask. In some embodiments, the mask 304 may be a silicon dioxide mask. In some embodiments, the mask 304 may be removed by etching. In some embodiments, the mask 304 may be removed by dry etching. In some embodiments, the mask 304 may be removed by chemical mechanical polishing (CMP). In such an embodiment, the deposited feature material 506 contacts the entire front side 311 as depicted in FIG. 5D. In such embodiments, the method may comprise an operation (e.g., operation (4)) comprising removing the deposited feature material by etching the front side until deposited material only resides in the cavity 305 as depicted in FIG. 5E. In other embodiments, the deposited material 506 may be removed by chemically mechanically polishing (CMP) the front side 311 until deposited material only resides in the cavity 305 as depicted in FIG. 5E. In some examples, operations (5)-(12) as described for FIGS. 3A-3M may be carried out on the substrate as depicted in FIG. 5E to yield the substrate (e.g., device 200) as depicted in FIG. 5F.

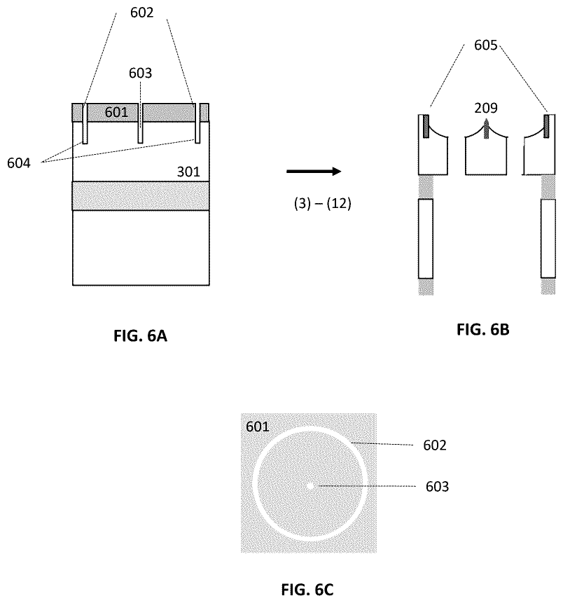

FIGS. 6A-6B show an abbreviated schematic for an exemplary fabrication process involving depositing a material that may be different from the substrate material to form both the projection 209 and well boundary perimeter 601 of a capture site in accordance with some embodiments. In this schematic, the substrate of FIG. 6A may be similar in processing state as the substrate of FIG. 3C. In some embodiments, the substrate of FIG. 6B may be similar in processing state to the substrate as depicted in FIG. 3M. Operations (3)-(12) as described in the description of FIGS. 3A-3M may be performed to transform the substrate as depicted in FIG. 6A to the substrate as depicted in FIG. 6B (e.g., device 200). In some embodiments, a mask 601 comprising openings 602 and 603 as depicted in FIG. 6A, may comprise a single annular opening 602 as depicted in a top-down view of the mask 601 in FIG. 6C. In some embodiments, etching through the opening 602 results in forming a circular trench within the substrate. In some embodiments, etching through the opening 603 results in a cylindrical cavity. In some embodiments, operations (3)-(12) as described within the description for FIGS. 3A-3M may be carried out resulting in the substrate 300 as depicted in FIG. 6B. In some embodiments, the annular opening 602 is configured to allow for etchant to form an annular cavity 604. The annular cavity upon filing with deposited feature material may be configured to form the well boundary perimeter 605, wherein the deposited feature material is different from the substate material.

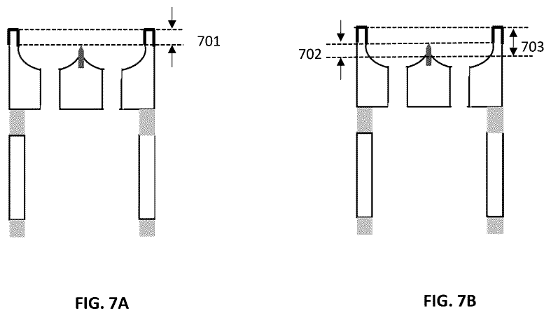

FIG. 7A depicts a recess gap 701 which refers to the distance between the tip of the projection 209 and the surface 202 of the device. Similarly, FIG. 7B depicts the projection length 702 and the well depth 703. The methods as described in the present application, comprising deposition of a material different from the substrate material, to form the projection 209, may allow for precise and accurate control of the projection length.

In some embodiments, methods as described in the present application allow for selecting a feature material (e.g., material to be deposited to form a projection and/or well boundary perimeter) that may be different from the substrate material. In some embodiments, the feature material may be insoluble in an etchant and the substrate material may be soluble in the same etchant.

In some embodiments, the patterning of the mask (e.g., masking layer) on the front side includes a photolithography and etching process as depicted in operations (1)-(12) of FIGS. 3A-3M. In some embodiments, the front side patterning process may be performed over the portion of the masking layer substrate to expose the features layer. In some embodiments, the masking layer may be etched in the shape of a projection. In some embodiments, the etching can be by dry etching (e.g., plasma etching), chemical mechanical polishing (CMP), or wet chemical etching. In some embodiments, a non-conductive or conductive layer may be formed on the surface of the substrate (e.g., semiconductor substrate) and can be deposited into the etched cavity 305. In some embodiments, the length (e.g., height) and recession (e.g., recess gap) of the projection can be varied and controlled by the deposited amount of the material. In some embodiments, the feature material (e.g., the fill material) can be deposited by atomic layer deposition (ALD), sputtering, physical vapor deposition (PVD), or other deposition processes. In some embodiments, the fill or deposited material can be silicon dioxide, aluminum dioxide, hafnium dioxide, titanium dioxide, chrome oxide, chromium oxide, zirconium oxide, zinc oxide, silicon nitride, tungsten, platinum, boron nitride, and titanium nitride. In some embodiments, the material (e.g., conductive material) may be etched off the front side leaving a cavity comprising conductive material. In some embodiments, the patterning of the features layer on the front side includes a photolithography and an etching process. In some embodiments, the openings of the mask (e.g., masking layer) may be etched in the shape of the ports (e.g., aspiration vias). In some embodiments, an isotropic etch may be performed into the features layer creating the well. An anisotropic etch may be performed into the features layer creating the port channels (e.g., aspiration via channels). In some examples, a dry etch may be used to remove the masking layer exposing the projection. In certain instances, the patterning of the masking layer on the backside may include a photolithography and etching process. The back side patterning process may be performed over the portion of the masking layer substrate to expose the backside of the features layer. In some embodiments, dry etching (e.g., plasma etching) from the backside may be performed covering each subarray and the resultant etch may remove a portion of the features layer and exposes the port channels. In some embodiments, the projection can be sharpened by Argon sputter etching, ion milling, thermal oxidation followed by buffered oxide etch or other process with little or no impact on well diameter.

In certain aspects, the patterning of the masking layer on the front side may include a photolithography and etching process as depicted in FIGS. 5A-5F and operations (1) through (12).

In some embodiments, depositing a material onto a substrate, through openings of a mask, allowing for deposition of material at select locations on the substrate is referred to as selectively depositing. In some embodiments, etching a substrate, through openings of a mask, allowing for etching of the substrate at select locations on the substrate is referred to as selectively etching.

### Method of Fabrication—Oxide-Based Device Layer

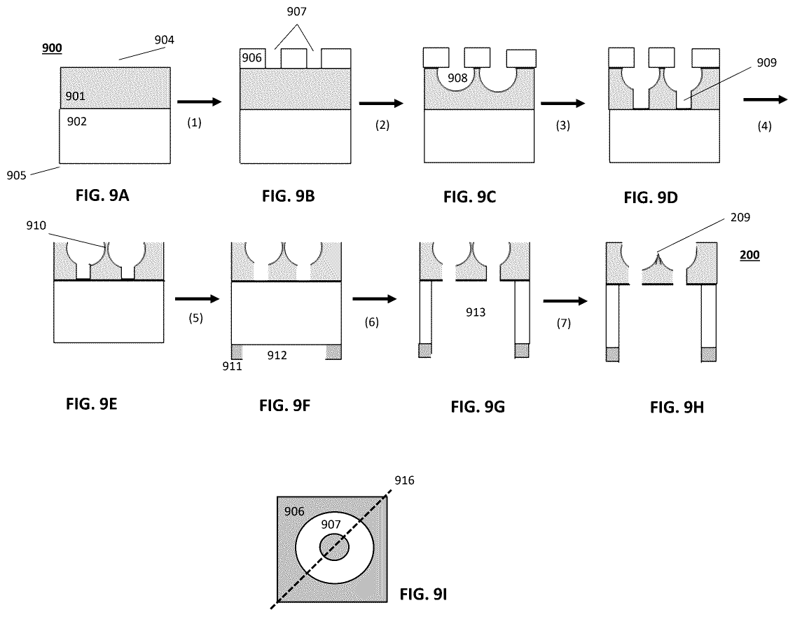

Described herein are various embodiments of a method for the fabrication of devices, wherein the devices may comprise a projection (e.g., microneedle) array. FIGS. 9A-9H provides cross sectional views of various aspects of the process of forming a projection-based device (e.g., microneedle), wherein the device may comprise a well, projection, and ports. In some embodiments, the substrate may comprise single or multiple patterning layers (e.g., device layers), masking layers (e.g., masks), oxide layers (e.g., oxide-based device layers), structural layers (e.g., handle layers) or a combination thereof. In some embodiments the layers of the substrate may comprise silicon in single crystal, polycrystalline, or amorphous structure. In some embodiments the layers can be made of silicon nitride, silicon dioxide, buried oxide, silicon carbide, gallium arsenide, gallium phosphide, indium phosphide, indium arsenide, a semiconductor alloy, or combination thereof. In some embodiments there can be more than one masking, oxide, and structural layers. The oxide layer can be formed by plasma, thermal oxidation, or chemical vapor deposition (CVD). The masking layer thickness can be about 0.1 microns to about 10 microns. The oxide layer thickness can be about 0.1 to about 100 microns. The structural layer thickness can be about 0.1 to about 1000 microns.

Described herein are various embodiments of a method for the fabrication of a device comprising an oxide device layer (e.g., feature layer). In some embodiments, the forming (e.g., patterning) of the mask (e.g., patterned masking layer) on the front side includes a photolithography and etching process as shown in FIGS. 9A-9H. In some embodiments, a substrate 900 may be provided comprising a device layer 901 and a handle layer 902. In some embodiments the device layer may comprise an oxide layer and the handle layer may comprise a silicon layer may be provided as depicted in FIG. 9A. In certain aspects, a mask 906 comprising an opening 907 may be formed in operation (1) on the front side 904 of the substrate, thereby yielding the substrate of FIG. 9B. In some embodiments, the opening 907 may be an annular opening as depicted in the mask 906 of FIG. 9I. The annular opening 907 intersects the cross section (e.g., dashed line of FIG. 9I) 916 twice, as depicted FIG. 9B. In some embodiments, the openings of the mask (e.g., masking layer) expose the oxide layer (e.g., features layer) as shown in FIG. 9B. In some embodiments, an isotropic etch may be performed in operation (2), resulting in a well 908 as shown in FIG. 9C. In certain aspects, the oxide layer 901 may be about 50 microns in thickness. In some embodiments, the oxide layer may comprise a thickness of about 1 micron to about 100 microns. In some embodiments, the oxide layer may comprise a thickness of about 1 micron or less. In some embodiments, the oxide layer may comprise a thickness of about 100 microns or more. In certain aspects, the oxide layer may comprise a thickness of less than 50 microns. In some embodiments, the oxide layer may be about 10 to about 100 microns thick. In some embodiments, the oxide layer may be formed by thermal oxidation of the substrate. In some embodiments, the oxide layer may be formed by a deposition process. In some embodiments, an etch may be performed in operation (3) of FIGS. 9A-9H that may be configured to etch anisotropically through the oxide layer. In some embodiments, the etch may be configured to etch the oxide layer over the silicon layer 902, thereby forming aspiration vias (e.g., aspiration via channels) 909 terminating at the silicon layer as shown in FIG. 9D. In some embodiments, the remaining substrate material at the center of the well may comprise a blunt, preliminary projection 910 as shown in FIG. 9C-9G. A dry etch in operation (4) of FIGS. 9A-9H may be used to remove the masking layer, resulting in the substrate shown in FIG. 9E. A back side 905 patterning process in operation (5) of FIGS. 9A-9H may result in a mask (e.g., masking layer) 911 with openings 912 as shown in FIG. 9F. In some embodiments, a plasma etch from the back side 905 may be performed in operation (6) of FIGS. 9A-9H forming a back side sub-array trench 913 that aligns with each sub-array 204 as shown in FIG. 9G. In some embodiments, the plasma etch of operation (6) may result in connecting the back side sub-array trench to the aspiration via channels. In some embodiments, operation (7) may comprise sharpening the blunt projection by argon sputter etching, ion milling, thermal oxidation followed by buffered oxide etch or other process with little or no impact on well diameter as shown in FIG. 9H. In some embodiments, in operation (7) wherein argon sputter etching, ion milling, or milling method may be used for sharpening the projection, the incident angle of the ions or physical etchant accelerated to the surface in the milling process may comprise an oblique angle to the front side of the substrate. In some instances, the oblique angle may be 45 degrees from orthogonal to the front side of the substrate. In some embodiments, wherein ion milling occurs at an oblique angle to the front side of the substrate, the substrate may be rotated about an axis that is orthogonal to the front side.

### Method of Manufacture for Surface Modification

Described herein are various methods comprising molecular and/or cell manipulation capabilities allowing for delivery, concentration, and/or mixing of cells and/or payloads (e.g., cargo) on or near the sub-array 204 of capture sites 205 before, during, and/or after payload insertion (e.g., transfection).

In some embodiments, the payload may be coated or adsorbed onto the device. In some embodiments, the device surface may be modified or coated with agents allowing for secondary payload coating or adsorption. In some embodiments, the coating method may comprise bulk adsorption, sputtering, evaporation, spotting, printing or electrodeposition methods.

### EXAMPLES

The following illustrative examples are representative of embodiments of the devices, systems, and methods described herein and are not meant to be limiting in any way.

### Example 1—Selective Material Deposition-Based Device Fabrication

This example describes a method as depicted in FIGS. 3A-3M for fabricating a device 200. A substrate 300 is obtained or provided, wherein the substrate has a device layer (e.g., feature layer) 301, an insulator layer (e.g., buried oxide layer or BOX layer) 302, a handle layer (e.g., structural layer) 303, a front side 311 and a back side 312 as depicted in FIG. 3A.

A mask 304 is formed (e.g., operation (1)) on the front side 311 of the substrate 300 of FIG. 3A, yielding the substrate 300 of FIG. 3B. The mask 304 may comprise an opening 313 exposing the device layer to etchants.

Anisotropic dry etching of the substrate 300 (e.g., operation (2)) as depicted in FIG. 3B, yields the substrate 300 comprising a feature region (e.g., cavity) 305 as depicted in FIG. 3C.

Depositing a material (e.g., operation (3)) through an opening 313 of the mask 304 of substrate 300 as depicted in FIG. 3C, yields the substrate as depicted in FIG. 3D comprising a deposited material 306.

The deposited material is removed by a lift-off process as depicted in FIGS. 4A-4B. In this example, operation (2) of FIGS. 4A-4C is not performed. A substrate 300 as depicted in FIG. 4A and equivalently in FIG. 3D, is provided. As depicted in FIG. 4A, the substrate 300 has a lift-off layer 401. The lift-off process (operation (1)) yields the substrate 300 of FIG. 4B and involves dissolving the lift-off layer 401 thereby lifting-off (e.g., removing) the mask 304 and the excess deposited material 306 residing on top of the mask 304. Both the substrate material and the deposited material are not soluble in the solvent used in the lift-off process (e.g., operation (1)) of FIGS. 4A-4C), therefore the deposited feature material 306 in the cavity 305 is retained. The lift-off process does not require the need to remove the mask 304 by etching or CMP and therefore the original high-quality factory surface of the substrate (e.g., wafer) is maintained. The advantage of not disturbing the original factory manufactured surface is that the low variation in flatness and low amount of roughness is maintained. In turn, the coefficient of variance (e.g., standard deviation/mean, % CV) in the recess gap and/or projection length-well depth ratio across all capture sites of the device is kept low resulting in higher precision and accuracy in the gene transfection process as depicted in FIGS. 1A-1F, across the device.

A mask 314 is formed on the front side of the substrate 300 (e.g., operation (5)) of FIG. 3E, yielding the substrate of FIG. 3F. The mask 314 comprises openings 315 exposing the device layer to etchants where the well of each capture site is to be formed.

The front side of the substrate is then isotropically wet etched yielding the substrate as depicted in FIG. 3G. Each opening 315 of the mask 314 allows for etchant to form a well 316 in the substrate. In this example, an initially annular shaped trench is expanded into a circular well with a projection at the center as depicted in the cross sections of FIGS. 3G and 8B. FIG. 8C shows a top-down view of the mask 801 and the annular opening 802 of the mask. The dashed-line 803 depicts the cross-section as viewed in FIGS. 3A-3M and 8A-8B.

The substrate is then anisotropically dry etched through circular openings of a port mask (e.g., aspiration via mask) comprising four openings per well to allow for four circular holes (e.g., ports, port channels or aspiration via channels) 208 to be etched in a square pattern within each well (e.g., operation (7)) of FIGS. 3A-3M. Operation (7) yields the substrate as viewed in FIG. 3H. The cross sections of the mask configured to form wells and the port mask configured to form aspiration via channels, look the same even though the opening patterns are different as viewed from the top-down.

The mask 314 is then removed by dry etching (e.g., operation (8)) yielding the substrate as depicted in FIG. 3I. Upon removal of the mask 314, a blunt projection 309 and a device surface 202 are present as depicted in FIG. 3I.

A mask 317 is then formed on the back side surface 312 of the substrate 300 of FIG. 3J (e.g., operation (9)). The mask 317 comprises an opening 318 exposing the handle layer (e.g., structural layer) 303. The opening 318 is aligned to a sub-array 204 on the front side 311 of the substrate.

A back side sub-array trench 310 is then dry etched into the handle layer 303 (e.g., operation (10)) yielding the substrate 300 as depicted in FIG. 3K.

The silicon dioxide insulator layer (e.g., BOX layer) 302 is then dry etched (e.g., operation (11)) resulting in connection of the aspiration via channels 208 on the front side of the substrate to the back side sub-array trench 310 yielding the substrate as depicted in FIG. 3L.

Following connection of the back side sub-array trench with the aspiration vias of the front side, a sharpening process (e.g., operation (12)) is then performed to transform the blunt projection 309 into the substrate comprising a projection 209 as depicted in FIG. 3M. Sharpening is performed by Argon sputter etching while Argon ions are directed to the surface of substrate at an angle of 45 degrees from vertical, while the substrate is rotating about the central vertical axis.

### Example 2—Silicon Dioxide Feature Layer-Based Device Fabrication Method

The patterning of the masking layer on the front side includes a photolithography and an etching process as shown in FIGS. 9A-9H. A substrate 900 comprising an oxide layer 901, a silicon layer 902, a front side 904 and a back surface 905 is provided as depicted in FIG. 9A. A well-mask 906 comprising a single annular opening 907 is formed in operation (1) on the front side 904 of the substrate 900 as depicted in FIGS. 9B and 9I. The opening exposes the oxide layer (e.g., features layer) 901. An isotropic etching process comprising a sodium hydroxide solution as the etchant is performed in operation (2), resulting in circular trench (e.g., well) 907 as shown in the cross-sectional view of FIG. 9C. The oxide layer in this example is 50 microns thick. Using a port mask (e.g., aspiration via mask) (as described in example 1) a dry etch is performed in operation (3) of FIGS. 9A-9H that is configured to etch anisotropically through the remaining depth of the oxide layer. Additionally, the etch is configured to etch the oxide layer over the silicon layer 902, thereby forming aspiration vias (e.g., aspiration via channels) stopping at the silicon handle layer as shown in FIG. 9D. A dry etch in operation (4) is used to remove the masking layer 906, resulting in the substrate shown in FIG. 9E. The resultant material at the center of the pattern forms a blunt, preliminary projection also shown in FIG. 9E. The back side patterning process of operation (5) results in a mask (e.g., masking layer) 911 with openings 912 as shown in FIG. 9F. A plasma etch from the back side is performed in operation (6) forming a back side sub-array trench 914 that aligns with each sub-array as shown in FIG. 9G. The plasma etch of operation (6) results in connecting the back side sub-array trench to the aspiration via channels. In operation (7) the blunt, preliminary, projection 910 is treated with argon sputter etching as described in example 1, forming a sharpened projection 209, with little or no impact on well diameter as shown in FIG. 9H.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure.

1. A method comprising the operations of:
a. etching a plurality of feature regions on a front side of a substrate, the substrate comprising a substrate material;
b. depositing a feature material on the front side of the substrate;
c. etching a portion of the deposited feature material on the front side of the substrate, thereby forming a front side surface;
d. etching the front side of the substrate to form a plurality of wells;
e. etching the front side of the substrate to form a plurality of ports; and
f. etching a back side of the substrate to form at least one sub-array trench, and
wherein the substrate material is different from the feature material.

2. The method of claim 1, wherein the depositing comprises atomic layer deposition (ALD), sputtering, physical vapor deposition (PVD), chemical vapor deposition (CVD), plasma-enhanced chemical vapor deposition (PECVD), electron beam evaporation, or thermal evaporation.

3. (canceled)

4. The method of claim 1, wherein the etching comprises etching the front side of the substrate until the deposited feature material is only present within the plurality of feature regions.

5. (canceled)

6. The method of claim 1, wherein the feature material comprises silicon dioxide, aluminum dioxide, hafnium dioxide, titanium dioxide, chrome oxide, chromium oxide, zirconium oxide, zinc oxide, silicon nitride, tungsten, platinum, boron nitride, porous silicon, or titanium nitride.

7-12. (canceled)

13. The method of claim 1, wherein the etching the front side of the substrate to form the plurality of wells comprises selective and isotropic wet etching through openings of a well-mask.

14. The method of claim 1, wherein the etching the front side of the substrate to form the plurality of ports comprises anisotropically etching the front side of the substrate.

15. The method of claim 1, wherein the etching the back side of the substrate to form the at least one sub-array trench comprises anisotropically etching the back side of the substrate.

16-18. (canceled)

19. The method of claim 1, wherein each feature region of the plurality of feature regions is configured to form a feature of a plurality of features of the device, wherein each feature of the plurality of features comprises a projection.

20-22. (canceled)

23. The method of claim 1, wherein forming the feature region comprises anisotropically etching the feature region with a dry etch process through a feature region-mask.

24-28. (canceled)

29. The method of claim 1, wherein the projection tip comprises a recess depth, the recess depth comprising the distance between the extreme end of the projection tip and the front side surface.

30-38. (canceled)

39. The method of claim 1, wherein the substrate comprises a feature layer, an insulator layer, a handle layer, or a combination thereof.

40. The method of claim 1, wherein the etching the back side of the substrate to form the at least one sub-array trench comprises aligning the at least one sub-array trench on the back side of the substrate to the at least one sub-array on the front side of the substrate.

41. The method of claim 1, wherein the etching the back side of the substrate to form the at least one sub-array trench comprises etching through the handle layer and the insulator layer, thereby allowing the plurality of aspiration via channels to be connected with the at least one sub-array trench.

42. (canceled)

43. The method of claim 1, further comprising functionalizing the projection with a payload.

44. The method of claim 43, wherein the payload comprises a biomolecule.

45-51. (canceled)

52. The method of claim 1, wherein operation (f) is performed prior to operations (a)-(d).

53. The method of claim 1, wherein operation (f) is performed after operations (a)-(d).

54-60. (canceled)

61. A device comprising:
a. a substrate comprising a surface; and
b. a capture site, the capture site comprising:
i. a well having a well boundary shape at the surface and having a well depth, the well configured to support a cell; and
ii. a projection extending from the bottom of the well toward the surface of the substrate, and
wherein the projection does not move relative to the substrate.

62. A device comprising:
a. a substrate comprising a surface; and
b. a capture site, the capture site comprising:
i. a well having a well boundary shape at the surface and having a well depth, the well configured to support a cell; and
ii. a projection extending from the bottom of the well toward the surface of the substrate, the projection comprising a projection length, and
wherein the substrate material is different than the feature material.

63. The device of claim 61, wherein the substrate comprises a feature layer, an insulator layer, and a handle layer, and wherein the capture site further comprises at least one aspiration via extending through the feature layer and fluidically connecting the well to a sub-array trench formed in the handle layer on a back side of the substrate.
